(19) United States
(12) Patent Application Publication
(10) Pub. No.: US 2005/0272967 A1
(43) Pub. Date: Dec. 8, 2005

(54) Biomolecular contrast agents with multiple signal variance for therapy planning and control in radiation therapy with proton or ion beams

(75) Inventors: Klaus Abraham-Fuchs, Erlangen (DE); Michael Moritz, Mistlegau (DE)

Correspondence Address: Siemens Corporation;Att: Elsa Keller, Legal Administrator, Intellectual Property Department, 170 Wood Avenue South, Iselin, NJ, 08830, US

(73) Assignee: Siemens Aktiengesellschaft

(21) Appl. No.: 10/848,880

(22) Filed: May 18, 2004

(51) Int. Cl. A61N 5/00

(52) U.S. Cl. 600/3

(57) ABSTRACT

A bio-molecular contrast agent (BMCA) is introduced into a biological organism such that the agent binds or reacts with target tissue within that organism. The BMCA is also signal-giving, allowing control of particle beam therapy by tracking the signal given by BMCA. The BMCA gives multiple signals which interact with different tissue elements in different ways. The variance in these signals is utilized to determine the constituency of the tissue either before and/or during therapy.

### CROSS-REFERENCE TO RELATED APPLICATIONS

This application is related to 1) a patent application entitled “Biomolecular Contrast Agents For Therapy Success And Dose Monitoring In Radiation Therapy With Proton Or Ion Beams” bearing attorney docket number 2004P01914US, filed concurrently herewith, and incorporated by reference herein; 2) a patent application entitled “Biomolecular Contrast Agents For Therapy Optimization In Radiation Therapy With Proton Or Ion Beams” bearing attorney docket number 2004P01915US, filed concurrently herewith and incorporated by reference herein; and 3) a patent application entitled “Biomolecular Contrast Agents For Therapy Control In Radiation Therapy With Proton Or Ion Beams” bearing attorney docket number 2003P19082US, filed concurrently herewith and incorporated by reference herein.

### BACKGROUND

1. Field of the Invention

This invention relates generally to the art of radiation therapy and diagnostic imaging. More specifically, the invention relates to the use of contrast agents in therapy planning and treatment involved in radiation therapy.

2. Related Art

In the treatment of cancer and other diseases, therapeutic measures such as particle beam therapy are commonly employed. In particle beam therapy, a beam (or beams) of radiation in the form of electrons, or photons, or more recently, protons, is delivered to a tumor or other target tissue. The dosage of radiation delivered is intended to destroy the tumorous cells or tissues.

It is state of the art today that medical imaging techniques such as CT (Computed Tomography), MR (Magnetic Resonance), PET (Positron Emission Tomography), optical imaging (ultraviolet/infrared/visible) or ultrasound are used to visualize the target region (most often a tumor) for particle beam therapy. Yet, the medical imaging techniques used for this purpose in many cases cannot reliably differentiate between malign tumors and benign tumors, and in particular are not well suited to visualize exactly the borderline between healthy tissue and malign tumors. Thus the therapy control methods today are based on non-optimal medical images, and as a consequence, for the sake of a successful destruction of the tumor, the volume to be irradiated usually is chosen larger than absolutely necessary thereby damaging healthy tissue in the process. Exact positioning and dosage is especially critical in therapies that use proton beams, where the energy is highly concentrated in particular locations due to the well-know Bragg Peak phenomenon.

Additionally, it happens in many cases that the images used for therapy planning do not exactly show the location of the target tissue for irradiation during the therapy session, for example because the patient is not positioned exactly in the same way during the imaging and the therapy session, or because the filling of the intestinal tract is different in both sessions, and thus organs are shifted. The composition and relative thickness of fatty tissue, fluids, muscle, and connective tissue in the beam pathway needs to be known, and unfortunately, can change after therapy planning. Recently, artificial or anatomical landmarks are used to control the position of the target tissue.

One solution that has been used recently in some imaging techniques is the introduction of “contrast agents” which enhance the image quality achieved during imaging. To provide diagnostic data, the contrast agent must interfere with the wavelength of radiation used in the imaging, alter the physical properties of the tissue/cell to yield an altered signal or provide the source of radiation itself (as in the case of radio-pharmaceuticals). Contrast agents are introduced into the body of the patient in either a non-specific or targeted manner. Non-specific contrast agents diffuse throughout the body such as through the vascular system prior to being metabolized or excreted. Non-specific contrast agents may for instance be distributed through the bloodstream and provide contrast for a tumor with increased vascularization and thus increased blood uptake. Targeted agents bind to or have a specific physical/chemical affinity for particular types of cells, tissues, organs or body compartments, and thus can be more reliable in identifying the correct regions of interest.

Several different targeted contrast agents which bind to particular tissue and then exhibit signal changes based upon state changes in tissues (which are then imaged) are disclosed in international patent application WO 99/17809, entitled “Contrast-Enhanced Diagnostic Imaging Method for Monitoring Interventional Therapies”.

In particular, the parameters of therapy that are planned for can often not be guaranteed to succeed nor be accurate due to changes in tissue state, position and surroundings. The methods used today in planning therapy and optimizing therapy in real-time during the therapy session, are sub-optimal and need to be improved.

### BRIEF DESCRIPTION OF THE DRAWINGS

FIG. 1 illustrates therapy optimization using BMCA according to one embodiment of the invention.

FIG. 2 illustrates an example of a tissue composition determination using multiple signal BMCAs in accordance with at least one embodiment of the invention.

FIG. 3 illustrates a system utilizing one or more embodiments of the invention.

### DETAILED DESCRIPTION OF THE INVENTION

In various aspects of the invention, bio-molecular contrast agents (BMCAs) are introduced into a patient for the purpose of radiation therapy planning and treatment. “BMCA”, as the term is used in describing this invention, are at least partially organic contrast agents which have the following properties: 1) they bind to target tissue, cells, and organs, and/or (2) react with metabolic products of the target tissue, cells, and organs by means of highly specific biochemical reactions (such as body-anti-body mechanisms). This yields an improved highly precise image of the target region for irradiation. In some embodiments, the invention also uses BMCA that are designed to have certain signal-giving properties as well as having a binding or reactive function. The reactive function can also activate the signal-giving property of the BMCA. These mechanisms help to ensure that the signals used for therapy planning, monitoring and control originate only from the target tissue.

For instance, fluorescent BMCAs, such as the ones described in U.S. Pat. No. 6,083,486, can be used in conjunction with a medical optical imager, like an optical tomograph or a diaphanoscope. As illustrated by the invention such BMCAs and other BMCAs can be adapted for use in therapy planning and real-time, on-line therapy control. One advantage of such BMCAs over conventional contrast agents is that the BMCAs stay immobilized for a longer period within the target tissue, due to the highly specific and stable binding reaction. Thus BMCAs are available for a longer time period to observe/monitor the target region than are conventional contrast agents.

BMCAs can also be designed or selected such that their signal-giving property diminishes when the BMCA interacts with the particle beam. The BMCA multiple signals can thus be “inactivated” (with respect to its signal-giving property) through irradiation with a particle beam of enough energy. For instance, a fluorescent contrast agent may be inactivated by destroying the fluorescence property of the BMCA which would involve breaking of the functional covalent C—C and/or C—H bindings of the BMCA through irradiation. In some embodiments of the invention, the beam energy, or respectively the irradiation dose, needed to inactivate the signal-giving property of the BMCA is roughly the same energy or dose as needed for successful medical treatment of the target tissue.

In this way, two types of information can be derived from the BMCA: the presence of the BMCA through specific binding indicates the target region for treatment while subsequent diminishing of the signal by destroyed signal-giving properties of the BMCA through the particle beam indicate that the target region has successfully been treated with the particle beam.

It is especially advantageous for the purpose of therapy optimization if the BMCA is designed such that the irradiation dose necessary the BMCA is designed such that the irradiation dose necessary to inactivate the BMCA corresponds roughly to the dose necessary to destroy DNA material in the target. Destruction of DNA material is one of the most important known mechanisms in the destruction of tumors through particle irradiation. In such a case, it can be assumed that the decrease of signal from the BMCA by interaction with particle beam is proportional to the degree of destruction of the tumor. To achieve this, in accordance with the invention, the BMCA is designed such that, in order to inactivate the signal-giving property of the BMCA, the destruction of one or more functional covalent C—C and/or C—H bindings (in the DNA) is necessary.

BMCA include small molecules and preferably bio-molecules with an affinity or reactivity with the target tissue. The affinity to bind or reactivity can be dependent on tissue state or tissue type or both. Bio-molecules are typically biologically derived or synthesized from naturally occurring elements such as amino acids, peptides, nucleotides and so on. Examples include receptor ligands, saccharides, lipids, nucleic acids, proteins, naturally occurring or genetically engineered anti-bodies. BMCA include those bio-molecules which can bind to proteins in plasma, in the fluid between cells, or in the space between cells. BMCA also includes dyes and other signal generating compounds, as desired. The difference in binding affinity of one bio-molecule versus another can have an effect in the signals that are ultimately received from the BMCA and in the accuracy of the binding to the target tissues. Thus, the specific nature and structure of the BMCA selected for the purpose of therapy control will depend upon which tissue or tissue component is to be bound. The binding sites for BMCA include such components and tissue as bones, calcified tissues, cancerous tissues, cell membranes, enzymes, fat, certain fluids (such as spinal fluid), proteins etc. BMCAs used in this invention may also include pharmaceutically accepted salts, esters, and derived compounds thereof, including any organic or inorganic acids or bases. BMCA may be accompanied by other agents, such as salts, oils, fats, waxes, emulsifiers, starches, wetting agents which may be used to aid in carrying the BMCAs to the target more rapidly or more securely, or in diffusing the BMCAs into external tissue such as skin.

During therapy planning prior to actual therapy, it is necessary to account for the thickness of the tissue in the particle pathway before the beam meets the target region. More specifically the relative thickness of fatty tissue, fluids, muscle and connective tissue in the beam pathway needs to be known. This information is conventionally derived from medical imaging (CT, MR, PET, ultrasound) and used in therapy planning algorithms. Yet, because of slightly different patient positioning, or shift/change in state of organs, tissue and fluid as compared to the imaging session, the composition of material in the particle beam pathway may have changed in the therapy session. In some embodiments of the invention, the BMCA signal decrease mechanism can be used for optimizing radiation dosage, energy and/or duration in order to achieve on-line, real-time therapy optimization. Thus, during irradiation, the parameters of therapy can be modified based upon feedback from the BMCA signals originating from the target tissue.

In one embodiment of the invention, a signal-giving BMCA is used, which delivers at least two different signals (e.g. fluorescence emission at two wavelengths), where the interaction properties (e.g. absorption of the signals by the tissue) with water and fatty tissue is different for the two signals. As an example, the contrast agent might contain fluorescent dyes with emission at two different wavelengths. The wavelengths of the fluorescent emission might be chosen such that absorption by fatty tissue is essentially the same at both wavelengths, but absorption by water is different at both wavelengths. In this case, the relative strengths of the signal at both wavelengths is proportional to the relative amount of water and fat in the signal pathway. The thus determined ratio of the amount of water and fat in the signal pathway is then compared to the ratio used in the model calculations for therapy planning. The therapy parameters can be adapted if the ratio has changed, either by outputting a corresponding recommendation to the operator, or by direct automated therapy device control.

This methodology can also be used to differentiate between other or more tissue types (blood, interstitial fluid, muscle, connective tissue, bone, fat etc.). This methodology implies that the pathway of the therapeutic particle beam and the pathway of the signals from the contrast agent see essentially the same or comparable body tissue distribution. The variance in BMCA signals at the different wavelengths will enable differentiation between tissue type and/or tissue composition. In other embodiments of the invention, a plurality of different BMCA can be introduced into a patient for the purpose of binding with the same target tissue. Each of the different BMCA will react with the beam in a different manner (for instance, be activated at different wavelengths).

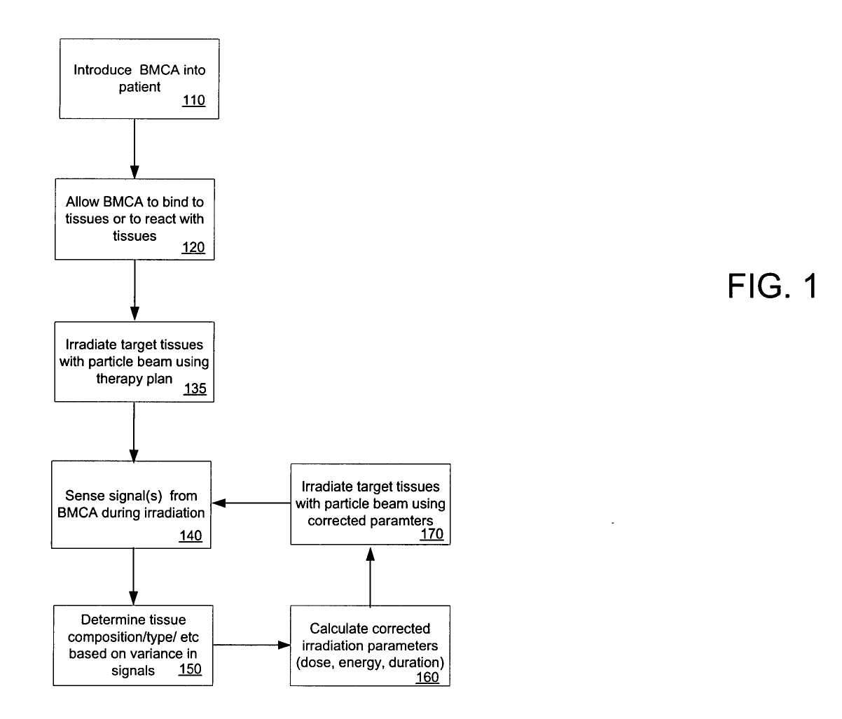

FIG. 1 illustrates therapy optimization using BMCA according to one embodiment of the invention. The patient (or other biological organism) is first subject to introduction of BMCA (block 110). Methods for introduction of BMCA may be similar to methods used to introduce other contrast agents, such as intravenous or oral and may be targeted or non-specific (such as those which spread throughout a region of the body). Other methods specific to BMCA may also be used. The BMCA, once introduced, is allowed to bind to tissues or react with the tissues (block 120). Thus, a suitable delay after introduction of the BMCA is required. This delay will vary based upon the type of binding or reaction, the type, size and location of the target tissue, the characteristics/affinity of the BMCA, and so on. The time for allowance should be sufficient to stabilize the BMCA binding or reaction with the target.

The BMCAs introduced according to block 110 give off multiple signals under different conditions. For instance, the BMCA can emit light in two or more different wavelengths. In such a case, the BMCA are chosen such that the interaction of signals can be different in the target depending upon what tissue property is being measured. For instance, the emitted signal can be absorbed at different levels as it is emitted from the BMCA and outside of the patient. If the tissue property were the thickness of various constituents of the tissue (such as fluid and muscle), the BMCA can be selected so that the absorption of the emitted signal is different in fluid versus muscle. This can be extended to any number of possible levels so that discrimination can be of many different tissue constituents. For instance, with the use of signals given off at three different wavelengths, it may be possible to distinguish between fluid, muscle as well as bone. The variance in signals can be measured and subjected to linear programming or other resolution mechanisms to determine the relative amounts (and, possibly, absolute amounts) of each constituent.

Next, the target tissues are irradiated with the particle/radiation beam (block 135) which may include any form of radiation including particle beams comprised of one or more of protons, electrons and photons. This irradiation is initially performed in accordance with pre-therapy plan models of the target tissue which includes tissue constituency. During irradiation, the strength of the signals from the BMCA is sensed continuously or at defined intervals (block 140). A detection/sensing system would capture the strength of the signals and convert them into a set of received signal values. The variance in received signal values are utilized to determine the constituency of the target tissue (block 150). The determination of tissue constituency (at block 150) will enable the calculation/computation of corrected therapy parameters, if any are required. The determined tissue constituency can be compared with tissue constituency models determined and utilized in pre-therapy planning. If the change is significant enough, corrected parameters of the therapy such as irradiation parameters of dose, energy, duration and so on, can be calculated (block 160). The corrected therapy parameters are then utilized, either automatically and/or manually, to modify the particle beam and irradiate the target using the corrected parameters (block 170). Any other changing constituency conditions can also be accounted for by continually monitoring the signals from the BMCA (block 140) and correlating and correcting as needed (blocks 150-170).

The exemplary workflow of FIG. 1 implies that a pre-therapy plan was derived using the then available tissue constituency. The variance in multiple BMCA signals is used to determine if the tissue constituency is different from that obtained in pre-therapy planning, hence potentially requiring a change in therapy parameters as well. This workflow might also include pre-therapy planning which is done according to conventional medical imaging and other conventional techniques (such as CT) and therapy optimization using multiple-signal BMCA during the therapy session in a real-time fashion. Other workflow possibilities include the use of multiple-signal BMCA immediately before the start of the therapy session (for instance, prior to the start of irradiation at block 135) to determine tissue constituency and modify the therapy plan prior to irradiation. This can as well be combined with the workflow for in-therapy therapy optimization using multiple-signal BMCA shown in FIG. 1 if needed or desirable. In addition, or in alternate, the multiple-signal BMCA can be administered and utilized during pre-therapy planning sessions to yield a potentially more accurate initial therapy plan. In still other embodiments of the invention, online therapy planning during the therapy session can be implemented by introducing the BMCA and beginning and optimizing the therapy during the therapy session.

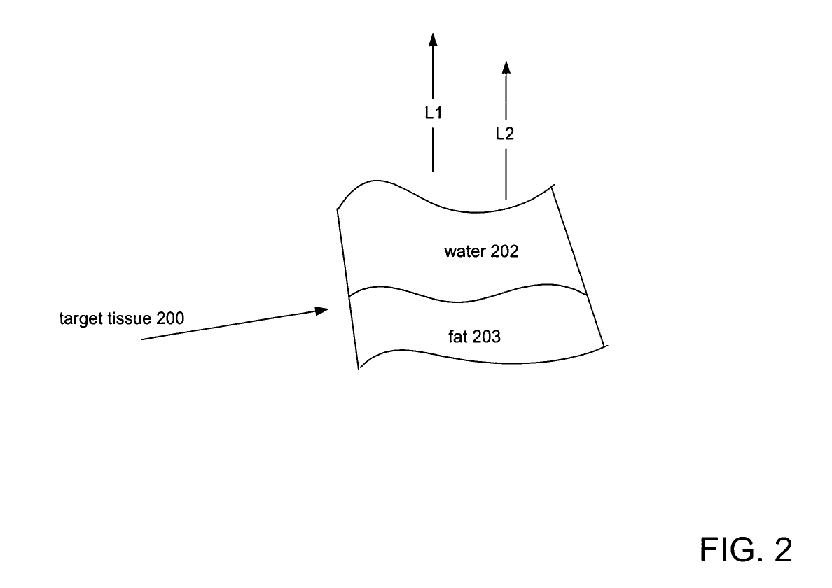

FIG. 2 illustrates an example of a tissue composition determination using multiple signal BMCAs in accordance with at least one embodiment of the invention. A target tissue 200 is composed, for instance, of water 202 as well as fat tissue 203. A signal-giving BMCA is introduced (not shown) and binds with tissue 200. The BMCA would give a signal, such as florescent emission, at two different wavelengths L1 and L2 (by the use for instance of dyes which emit at these wavelengths L1 and L2). The emitted fluorescent signals would have its energy absorbed in a different manner when impacting water 202 and when impacting the fat tissue 203. For instance, BMCA can be chosen/designed such that the absorption of signal energy in the fat tissue 203 could be similar at wavelength L1 and wavelength L2. In contrast, the absorption of emitted signal energy in water 202 could be different at wavelength L1 and wavelength L2. The signals from these emissions would be sensed or detected with the total received signal at wavelength L1 being designated TR_{L1} and the signal at wavelength L2 being designated as TR_{L2}. The ratio of the received signals in comparison to the total possible emitted signal can be used to determine the relative thicknesses of water and fat in the target tissue 200.

For instance, assume that the total possible emitted signal, that is the signal seen emitted at the source of the BMCA is TE_{L1} at wavelength L1 and TE_{L2} at wavelength L2. The signal received (sensed) is a fraction of the total emitted signal which has not been absorbed in the signal pathway back to the sensor/receiver. As assumed above, the total absorption of signal in fat is the same for both wavelengths. Hence, the received signals accountable to fat R_{L1}(fat) and R_{L2}(fat) is the same at both wavelengths. The received signals accountable to water R_{L1}(water) and R_{L2}(water) are different at both wavelengths. By measuring the total received signals TR_{L1} (=R_{L1}(water)+R_{L1}(fat)) and TR_{L2} (=R_{L2}(water)+R_{L2}(fat)) at the wavelengths L1 and L2, respectively, linear algebra can be used to resolve the amount of the signal due to water and due to fat, and hence the relative ratio for these can be established.

Specifically, the relative amount of water in the target tissue can be determined by the ratio:
Fraction Water=|TR_{L2}−TR_{L1}|/|TE_{L2}−TE_{L1}| (1).
The fraction of tissue due to fat is then determined by:
Fraction Fat=1−Fraction Water (2).

If there is no water and 100% fat content in the target tissue, then TR_{L2}−TR_{L1} would be zero, meaning that the received signal at both wavelengths are the same and keeping with the initial BMCA design, this correlates well with the fact that the absorption of signal is the same at both wavelengths for fat. This indicates that all of the signal received went through a pathway consisting of fat (provided that the received signal path is the same as the tissue pathway). If there were any water present, then the TR_{L2} would not be equal to TR_{L1}.

To take numerical example, assume that TE_{L2} is 120 units and TE_{L1} is 90 units. Also, assume that at 100% fat content R_{L1}(fat) and R_{L2}(fat) would be 30 units, while at 100% water content R_{L1}(water) is 60 units and R_{L2}(water) 90 units. If TR_{L2} is 70 and TR_{L1} is 50, then Fraction Water would be |70−50|/|120−90| or 2/3. This leaves 1/3 as the Fraction Fat, or percentage of tissue due to fat. This also corresponds exactly with the ratios 1/3*30+2/3*60=50 at wavelength L1 and 1/3*30+2/3*90=70 at wavelength L2.

The above example is one of many possible techniques to compute the relative thicknesses of different constituents in a target tissue. Other algorithms may include simple ratio tests, non-linear modeling, regression, and any such techniques designed to resolve unknown quantities. This idea can be extended to differentiating one type of tissue from another. For instance, the ratio of muscle to connective tissue can be determined by selecting BMCA which give signals that interact (absorb) differently in connective tissue and in muscle. In addition, the number of signals can be extended to three, four or any desired number in order to resolve three, four or any number, respectively, of tissue constituents or properties or conditions.

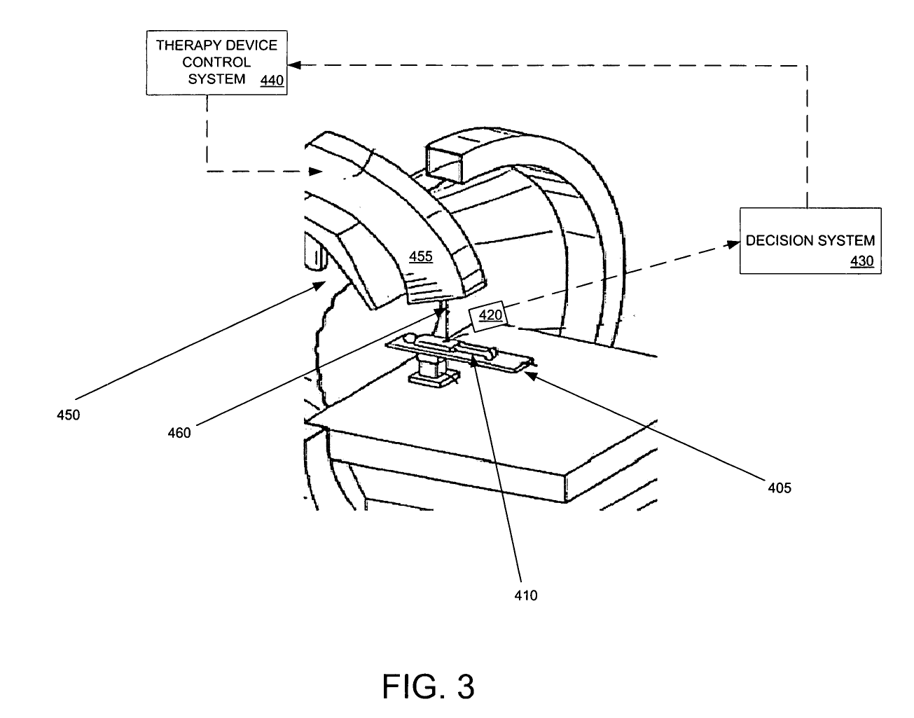

FIG. 3 illustrates a system utilizing one or more embodiments of the invention. At least a portion of a treatment room 400 is shown which houses a therapy device 450 and bed 405 which positions a patient 410 for treatment by treatment device 450. Treatment device 450 may be a radiation or energy delivery system such as proton or photon particle beam delivery system. Treatment device 450 may include a gantry (pictured but not enumerated) and treatment head 455. Treatment head 455 is responsible primarily for delivering and directing the desired or planned energy to patient 410 in the form of a beam 460, for instance. Treatment head 455 may include a number of different elements include scattering elements, collimators, boluses, refraction/reflection elements, and so on.

Generally, in the case of a beam 460 which is composed of particles (such as photons, protons, electrons, neutrons and heavy ions), a particle stream is externally generated and accelerated (by a cyclotron and/or linear accelerator) and then the particle stream (or a portion of it) is delivered to treatment head 455. Treatment head 455 can limit or define both the size and shape of the beam 460 as well as the intensity of the beam 460. Treatment head 455 may also contain a nozzle which can be rotated in different axes to deliver the beam 460. Utilizing this nozzle and various elements within the treatment head 455, therapy device 450 can deliver energy into patient 410 at a different incident angle and with varying shape, size and intensity, as desired. A therapy device control system 440 may be employed for the purpose of controlling the various elements of the treatment head 455 and for controlling the level of energy introduced from the externally generated particle source.

As mentioned above, linear programming models and variable resolution algorithms can be developed for tissue properties, conditions, constituency, etc. These models and algorithms may be made available as part of a decision system 430 or externally and made available thereto via a network or other communication means. Additional mechanisms such as models, software, neural networks and the like which assist in determining how conditions have changed and what therapy optimizations are desirable can be again part of the decision system 430 or separate but accessible thereto.

A therapy plan is ordinarily generated prior to actual therapy beginning. This therapy plan may have been based upon assumptions of certain parameters of the patient 410 such as the size and location of the target tissue, the composition of the target tissue, the composition of the pathway to the target tissue through the body, the state of the tissue and so on. The therapy plan may include parameters such as the geometry and location of the target tissue, marking the body, pre-therapy imaging of the target tissue, dosage plans, and the like. Since the pre-therapy plan is based upon currently available information on target tissue and related conditions, such conditions may change by the time treatment is actually commenced. In addition, it possible that conditions did not necessarily change but were misdiagnosed due to faulty data, faulty interpretation, etc. In such cases as well, the pre-therapy treatment plan may be inaccurate. In some embodiments of the invention, the pre-therapy planning process including imaging of tissue can be assisted by the use of multiple-signal BMCA. As discussed above, multiple-signal BMCA can be designed so that different interactions are possible with different types, conditions and states of target tissue and body pathways. The differences in these interactions can be utilized to resolve unknown types, conditions, and states of target tissue and body pathways so that a more accurate pre-therapy plan is possible.

In accordance with other embodiments of the invention, on-line therapy planning and therapy optimization is possible using the same or similar multiple-signal BMCA techniques. Prior to treatment by treatment device 450, multiple-signal BMCA is introduced into patient 410. The multiple-signal BMCA is given time to bind or react to target tissue within the patient 410 to which the beam 460 is to be directed. The therapy device control system 440 utilizes the therapy plan to direct beam 460 towards patient 410. This begins irradiation of the target tissue. In other embodiments of the invention, the BMCA can be used to initially direct beam 460 to the target tissue and planning and on-going optimization of treatment can be attained by use of the same or similar multiple-signal BMCA.

During irradiation, the conditions of the target tissue can be tracked by a sensing system 420. Sensing system 420 will be capable of receiving or detecting the signals emitted by the signal-giving properties of the multiple-signal BMCA which is bound to the target tissue within patient 410. Sensing system 420 may be, for example, an optical tomography device or a diaphonoscope which can detect the fluorescence given off the BMCA. The signals emitted by the BMCA may be optical, ultraviolet, infrared, electromagnetic (in the case of a radio-pharmaceutical BMCA), and so on. Sensing system 420 will be configured/designed to detect BMCA signals at different wavelengths or with different properties as such properties are used to distinguish one of the multiple signals for other ones. Sensing system 420 will be designed/selected in order to detect these signals and transfer this sensor data to decision system 430. Sensing system 420 may also include a source (not pictured) such as X-ray source in the case of simple X-ray imaging. Sensing system 420 will be able detect the presence and strength of the BMCA signals emitted from the target tissue within patient 410. This data can be utilized to determine if any tissue constituency, conditions, states, properties etc. of the patient 410 vary from that ascertained in pre-therapy planning or from the ongoing current treatment such that treatment must be changed or optimized. While sensing system 420 is pictured as a non-integrated unit, it can be integrated with the treatment head 455, if desirable, or positioned or integrated anywhere on the therapy device 450 as appropriate.

In some embodiments of the invention, the BMCA signal can be inactivated by exposure to beam 460. In such instances, the sensing system will detect the strength of the BMCA signal as an indication of impaction of beam 460 with the target tissue. In response to data received from sensing system 460, decision system 430 will be configured to determine any change in conditions, constituency, properties etc. of the target tissue. Decision system 430 may also have access to a pre-therapy planning data and images of the target tissue, if needed for additional analysis. Decision system 430 will determine if there is a change in the target tissue based upon variances in the multiple BMCA signal values. If there is, and this change is significant enough to affect the outcome of the current therapy, or if the change would indicate a change in the therapy plan, then decision system 430 can indicate these changes to the therapy device control system 440. Based upon these changes, the therapy device control system 440 can change the dosage, duration or positioning parameters of the beam 460 to resolve the change or variance conditions of the target tissue. The beam 460 can be also stopped altogether, if necessary, particularly if the sensing system 420 and decision system 430 indicate that the target tissue is no longer present. The decision system 430 may send condition change and/or resolution information to an operator which can then manually implement the modified therapy parameters to the therapy device control system 440 if deemed necessary. In other embodiments of the invention, the changes in operation of the therapy device control system 440 can be automated, whichever is more desired. In other embodiments of the invention, the therapy device control system 440 could modify the position of the patient 410 or the bed 405 in response to decision system 430 indicating a change in conditions of the target tissue.

For instance, assume a therapy plan assumed that the target tissue ½ fluid and ½ tumorous tissue (a 1:1 ratio) and therapy device control system 440 directed treatment by treatment device 450 on this basis. Multiple signals from BMCA can be designed such that their interaction with fluid and tumorous tissue is different. This variance can be used to determine the relative amount of actual tumorous tissue and fluid in the target. If the multiple signals given by the BMCA indicate for instance that the actual ratio of fluid to tumorous tissue were 1:2 rather than 1:1, then the irradiation depth of the beam 460 may have to be modified to deliver more energy to the parts of the target tissue that contain tumorous tissue which were not previously irradiated. If the beam 460 delivered radiation to the bottom ½ of the target tissue, then presumably ⅙ of the original tumorous may be irradiated insufficiently or not at all. The therapy can then be optimized, based upon this information, to irradiate the remaining ⅙ of the target tissue. This would include the sensing system 420 detecting the two signals from the BMCA and decision system 430 interpreting these signal values to arrive at the relative ratio of fluid to tumorous tissue of 1:2. The decision system 430 relays this data and/or an indication that another ⅙ of the target tissue has not been irradiated properly to therapy device control system 440. Therapy device control system 440 then directs or is controlled by an operator (who is aware of the decision system 430 indications) to direct treatment device 450 to modify the energy of beam 460 so that the remaining ⅙ of the target which is determined to be tumorous is treated appropriately.

The systems mentioned in the above description including the sensing system 420, decision system 430 and therapy device control system 440 may be any combination of hardware, software, firmware and the like. Further, all of these systems may be integrated onto the same hardware platform or exist as software modules in a computer system or both. The systems may be distributed in a networked environment as well and may be stand-alone components. One or more of the systems 420, 430 and 440 may be integrated with the therapy device 450 itself, or separate therefrom. Further, any number of these systems 420, 430 and 440 may be physically separated from the therapy device and manually/automatically monitored or controlled. Systems 420, 430 and 440 may utilize or be loaded into processors, storage devices, memories, network devices, communication devices and the like as desired. Sensing system 420 may also contain cameras, sensors, and other active/passive detection and data conversion components, without limitation.

While the embodiments of the invention are illustrated in which it is primarily incorporated within a radiation therapy system, almost any type of medical treatment of imaging system may be potential applications for these embodiments. Further, the bio-molecular contrast agents used in various embodiments may be any organic or semi-organic compounds which have the desired effect of affinity to certain target tissues/cells to either bind with them or react with them. The examples provided are merely illustrative and not intended to be limiting.

1. A method for treating a target within a biological organism with a beam of energy, said method comprising:
introducing a bio-molecular contrast agent (BMCA) into said biological organism, said BMCA capable of at least one of binding to said target and reacting with said target, said BMCA capable of also giving a plurality of detectable signals, each of which interact differently with at least one of said organism and said target;
irradiating said target using said beam of energy in accordance with a pre-therapy plan; and
after said BMCA has bound or reacted to said target, modifying parameters of said treating if said plurality of signals indicate that conditions of said target or said biological organism are sufficiently different enough from that assumed in said pre-therapy plan so as to require a change in said treating.

2. A method according to claim 1 wherein said target is a tissue in a particular state.

3. A method according to claim 1 further comprising:
sensing of said plurality of signals.

4. A method according to claim 1 wherein said conditions include at least one of the constituency of said target, the constituency of a pathway within said biological organism to said target, and the state of said target.

5. A method according to claim 3 wherein said sensing is performed using an imaging technique, further said plurality of signals are capable of being imaged.

6. A method according to claim 5 wherein said imaging technique is at least one of optical imaging, positron emission tomography, magnetic resonance imaging, X-ray imaging, ultrasound imaging and computed tomography.

7. A method according to claim 1 wherein said plurality of signals include at least one of fluorescence, luminescence and phosphorescence.

8. A method according to claim 1 further comprising:
utilizing the variance in said plurality of signals to detect changes of conditions of said target.

9. A method according to claim 6 wherein said optical imaging includes detecting at least one of visible, infrared and ultraviolet signals given by said BMCA.

10. A method according to claim 8 wherein each of said plurality of signals fluoresce at different wavelengths.

11. A method according to claim 1 wherein said beam of energy is composed at least one of proton, photon, heavy ion, neutron and electron particles.

12. A method according to claim 8 wherein said conditions include the relative amounts of constituent elements of said target.

13. A method according to claim 12 wherein said utilizing includes determining if the ratio of said constituent elements is sufficiently different from said pre-therapy plan.

14. A method according to claim 12 wherein each of said plurality of signals interacts differently with said constituent elements.

15. A method according to claim 1 wherein said pre-therapy plan is developed with the assistance of multiple-signal BMCA.

16. A method according to claim 1 wherein said pre-therapy plan is developed by techniques that do not use BMCA.

17. A method for treating a target within a biological organism with a beam of energy, said method comprising:
introducing a bio-molecular contrast agent (BMCA) into said biological organism, said BMCA capable of at least one of binding to said target and reacting with said target, said BMCA capable of also giving a plurality of detectable signals, each of which interact differently with at least one of said organism and said target;
irradiating said target using said beam of energy as indicated by said plurality of detectable signals after said BMCA has bound or reacted to said target, said irradiating performed without reference to pre-therapy planning; and
modifying said irradiating if said plurality of signals indicate that conditions of said target or said biological organism are sufficiently different during irradiating so as to require a change in said irradiating.

18. A method according to claim 17 wherein said target is a tissue in a particular state.

19. A method according to claim 17 further comprising:
sensing of said plurality of signals.

20. A method according to claim 17 wherein said conditions include at least one of the constituency of said target, the constituency of a pathway within said biological organism to said target, and the state of said target.

21. A method according to claim 19 wherein said sensing is performed using an imaging technique, further said plurality of signals are capable of being imaged.

22. A method according to claim 21 wherein said imaging technique is at least one of optical imaging, positron emission tomography, magnetic resonance imaging, X-ray imaging, ultrasound and computed tomography.

23. A method according to claim 17 wherein said plurality of signals include at least one of fluorescence, luminescence and phosphorescence.

24. A method according to claim 17 further comprising:
utilizing the variance in said plurality of signals to detect changes of conditions of said target.

25. A method according to claim 22 wherein said optical imaging includes detecting at least one of visible, infrared and ultraviolet signals given by said BMCA.

26. A method according to claim 24 wherein each of said plurality of signals fluoresce at different wavelengths.

27. A method according to claim 17 wherein said beam of energy is composed at least one of proton, photon, heavy ion, neutron and electron particles.

28. A method according to claim 24 wherein said conditions include the relative amounts of constituent elements of said target.

29. A method according to claim 28 wherein each of said plurality of signals interacts differently with said constituent elements.

30. A method for developing a pre-therapy treatment plan for treating a target within a biological organism with a beam of energy, said method comprising:
introducing a bio-molecular contrast agent (BMCA) into said biological organism, said BMCA capable of at least one of binding to said target and reacting with said target, said BMCA capable of also giving a plurality of detectable signals, each of which interact differently with at least one of said organism and said target; and
after said BMCA has bound or reacted to said target, deriving treatment plan parameters by detecting said plurality of signals, said signals indicating conditions of said target and said organism.

31. A method according to claim 30 wherein said target is a tissue in a particular state.

32. A method according to claim 30 further comprising:
sensing of said plurality of signals.

33. A method according to claim 30 wherein said conditions include at least one of the constituency of said target, the constituency of a pathway within said biological organism to said target, and the state of said target.

34. A method according to claim 32 wherein said sensing is performed using an imaging technique, further said plurality of signals are capable of being imaged.

35. A method according to claim 34 wherein said imaging technique is at least one of optical imaging, positron emission tomography, magnetic resonance imaging, X-ray imaging, ultrasound and computed tomography.

36. A method according to claim 30 wherein said plurality of signals include at least one of fluorescence, luminescence and phosphorescence.

37. A method according to claim 30 further comprising:
utilizing the variance in said plurality of signals to detect changes of conditions of said target.

38. A method according to claim 35 wherein said optical imaging includes detecting at least one of visible, infrared and ultraviolet signals given by said BMCA.

39. A method according to claim 37 wherein each of said plurality of signals fluoresce at different wavelengths.

40. A method according to claim 30 wherein said beam of energy is composed at least one of proton, photon, heavy ion, neutron and electron particles.

41. A method according to claim 37 wherein said conditions include the relative amounts of constituent elements of said target.

42. A method according to claim 41 wherein each of said plurality of signals interacts differently with said constituent elements.

43. A method for treating a target within a biological organism with a beam of energy, said method comprising:
introducing a plurality of bio-molecular contrast agents (BMCAs) into said biological organism, said BMCAs capable of at least one of binding to said target and reacting with said target, each of said BMCAs capable of also giving detectable signals which are distinguishable from signals of other of said BMCAS, each of said detectable signals interacting differently with at least one of said organism and said target;
irradiating said target using said beam of energy in accordance with a pre-therapy plan; and
after said BMCAs have bound or reacted to said target, modifying parameters of said treating if said plurality of signals indicate that conditions of said target or said biological organism are sufficiently different enough from that assumed in said pre-therapy plan so as to require a change in said treating.

44. A method according to claim 43 wherein said target is a tissue in a particular state.

45. A method according to claim 43 further comprising:
sensing of said detectable signals.

46. A method according to claim 43 wherein said conditions include at least one of the constituency of said target, the constituency of a pathway within said biological organism to said target, and the state of said target.

47. A method according to claim 45 wherein said sensing is performed using an imaging technique, further said plurality of signals are capable of being imaged.

48. A method according to claim 47 wherein said imaging technique is at least one of optical imaging, positron emission tomography, magnetic resonance imaging, X-ray imaging, ultrasound and computed tomography.

49. A method according to claim 43 wherein said detectable signals include at least one of fluorescence, luminescence and phosphorescence.

50. A method according to claim 43 further comprising:
utilizing the variance in said detectable signals to detect changes of conditions of said target.

51. A method according to claim 48 wherein said optical imaging includes detecting at least one of visible, infrared and ultraviolet signals given by said BMCAs.

52. A method according to claim 50 wherein each of said detectable signals fluoresce at different wavelengths.

53. A method according to claim 43 wherein said beam of energy is composed at least one of proton, photon, heavy ion, neutron and electron particles.

54. A method according to claim 50 wherein said conditions include the relative amounts of constituent elements of said target.

55. A method according to claim 54 wherein each of said detectable signals interacts differently with said constituent elements.
